(19) United States
(12) Patent Application Publication
(10) Pub. No.: US 2015/0342465 A1
(43) Pub. Date: Dec. 3, 2015

(54) METHOD OF DETERMINING TEMPERATURE SHIFTING ERROR DERIVED FROM RADIATION SENSOR, METHOD OF MEASURING OCULAR SURFACE TEMPERATURE AND APPARATUS THEREOF

(71) Applicant: UNITED INTEGRATED SERVICES CO., LTD., TAIPEI COUNTY (TW)

(72) Inventors: O CHANG, NEW TAIPEI CITY (TW); CHUNG-HWA CHANG, NEW TAIPEI CITY (TW); PO-HSUAN LIU, NEW TAIPEI CITY (TW); HUI-HUA KENNY CHIANG, TAIPEI CITY (TW)

(73) Assignee: UNITED INTEGRATED SERVICES CO., LTD., TAIPEI COUNTY (TW)

(21) Appl. No.: 14/287,357

(22) Filed: May 27, 2014

(51) Int. Cl. A61B 5/01; A61B 5/00; A61B 3/10

(52) U.S. Cl. CPC A61B 5/01; A61B 3/101; A61B 5/702; A61B 5/742; A61B 5/741; A61B 5/7221

(57) ABSTRACT

A method of determining temperature shifting error derived from radiation sensor comprising: providing an infrared heat sensor and a temperature sensor contacting with a black body, the temperature sensor measuring an actual black body temperature, the infrared heat sensor detecting a radiation emitted by the black body, the radiation being computed according to a temperature rising curve through a computing unit of a work station to generate a computed black body temperature; and determining a value of temperature shifting error by subtracting the actual black body temperature from the computed black body temperature. Accordingly, method of measuring ocular surface temperature and apparatus thereof based on the above shift error determining method are also provided.

### BACKGROUND

1. Field of the Invention

The instant disclosure relates to a method of determining temperature shifting error derived from radiation sensor, a method of measuring ocular surface temperature and the apparatus thereof; in particular, to a method of determining temperature shifting error by using black body that plays a calibration reference role for measuring ocular temperature and apparatus thereof.

2. Description of Related Art

Conventional method to measure tear break up of dry eye includes intrusive or non-intrusive approach. The intrusive approach includes Schirmer and tear break up time (TBUT). Take Schirmer for example. The eyes undergo anesthesia and an elongated filter paper is put under the lower eyelid, such that the filter paper absorbs tears from the ocular surface by capillary. After a few minutes, the amount of tear can be assessed by looking at the diffusion area caused by the tear absorbed by the filter paper. The TBUT is conducted by dropping fluorescence dye into the tester's eyes, and the first tear break up timing is observed by slit lamp. However, the abovementioned approaches includes eye contact with foreign subjects. In addition to uncomforting to the eyes, when conducting the abovementioned measure, because of the irritation to eye, the kinetics of tear break up may be altered and lead to reflective tearing. Therefore, the test result is not reliable and less reproducible. Furthermore, the result cannot achieve the idea measurement regulation proposed by the International Dry Eye Association in the 2007 annual report. The regulation said, when examining ocular surface tear change, the kinetics to the tear should be least affected.

The non-intrusive examination is primarily done by an ophthalmologist observing the tear break up of a tester and by infrared heat sensor to detect the ocular surface temperature change so as to lean the condition of tear break up. However, by looking into a patient's eyes, the result heavily relies on the clinical experience of the ophthalmologist, and there is not a quantified standard. Different doctors may have different interpretation of tear break up, and therefore misjudgment happens frequently. Especially, the degree of tear break up is the basis to determine dry eye or not, so the misjudgment is likely to happen. The infrared detection suffers from inaccuracy because the infrared sensor unit is highly sensitive to the ambient radiation (heat source). If there are other people around the tester, it becomes a source of external heat and the temperature detected by the infrared sensor drifts as well. Therefore, before the tester undergoes the examination, a 5- to 10-minute waiting time is required so as to achieve heat balance with the ambience and reduce error rate. However, in practical, the waiting time can hardly be enough when there are so many patients, and the error rate is usually high. The infrared examine apparatus can only detect relative temperature change but not an absolutely ocular surface temperature or other information related to the eye. Therefore the conventional infrared sensor has considerable error rate, and it cannot obtain complete data which helps the physician to determine the eye condition. The usage of the infrared sensor is relatively limited.

To address the above issues, the inventor strives via associated experience and research to present the instant disclosure, which can effectively improve the limitation described above.

### BRIEF SUMMARY OF THE INVENTION

The instant disclosure provides a method of determining temperature shifting error derived from radiation sensor to reduce error rate, comprising: (A) providing an infrared heat sensor and a temperature sensor contacting with a black body, the temperature sensor measuring an actual black body temperature, the infrared heat sensor detecting a radiation emitted by the black body, the radiation being computed according to a temperature rising curve through a computing unit of a work station to generate a computed black body temperature; and (B) determing a value of temperature shifting error by subtracting the actual black body temperature from the computed black body temperature.

The instant disclosure also provides a method of measuring ocular surface temperature basically derived from the above method for determining temperature shifting error to provide highly accurate data and result. The method of measuring ocular surface temperature includes the following steps: (a) detecting an ocular surface radiation emitted from a ocular surface of a tester by the infrared heat sensor, the ocular surface radiation being computed according to the temperature rising curve through the computing unit of the work station to generate a computed ocular surface temperature; (b) generating a calibrated ocular surface temperature by subtracting the value of temperature shifting error from the computed ocular surface temperature.

The instant disclosure also provides an apparatus to measure ocular surface temperature. According to one embodiment of the instant disclosure, the apparatus includes a supporting body, an infrared heat sensor, a black body, a temperature sensor and a working station. The supporting body supports a head, and the infrared heat sensor is disposed opposite to the supporting body such that the supporting body is positioned in a predetermined view field of the infrared heat sensor. The black body is disposed on the supporting body and in the predetermined view field. The temperature sensor is buried in and contacted with the black body, wherein the temperature sensor measures an actual black body temperature from the black body. The working station is electrically connected to the infrared heat sensor and has at least a computing unit and a storage unit. The infrared heat sensor detects a value of radiation emitted from the black body and the computing unit transfers the value of radiation into a computed black body temperature according to a temperature rising curve. Then, a value of temperature shifting error is determined by subtracting the actual black body temperature from the computed black body temperature.

In summary, the black body is the basis for calibration reference temperature, and the presence of the black body avoids ambient interference like heat to the infrared heat sensor, such that the temperature drifting of the infrared heat sensor may be reduced.

In order to further understand the instant disclosure, the following embodiments are provided along with illustrations to facilitate the appreciation of the instant disclosure; however, the appended drawings are merely provided for reference and illustration, without any intention to be used for limiting the scope of the instant disclosure.

### BRIEF DESCRIPTION OF THE SEVERAL VIEWS OF THE DRAWINGS

FIG. 1 is a flow chart showing a method of determining temperature shifting error derived from radiation sensor of the instant disclosure;

FIG. 2A is a flow chart showing a method of measuring ocular surface temperature of the instant disclosure;

FIG. 2B is a flow chart showing a method of measuring ocular surface temperature of the instant disclosure;

FIG. 3 is a perspective view of an apparatus to measure ocular surface temperature of the instant disclosure;

FIG. 4 is a schematic diagram showing a method of measuring ocular surface temperature and an apparatus thereof of the instant disclosure in operation;

FIG. 5 shows a temperature changing curve obtained from a method of measuring ocular surface temperature and a apparatus thereof of the instant disclosure in a unit time; and

FIG. 6 shows a temperature rising curve according to the black body radiation obtained from a method of measuring ocular surface temperature of the instant disclosure.

### DETAILED DESCRIPTION OF THE INVENTION

The aforementioned illustrations and following detailed descriptions are exemplary for the purpose of further explaining the scope of the instant disclosure. Other objectives and advantages related to the instant disclosure will be illustrated in the subsequent descriptions and appended drawings.

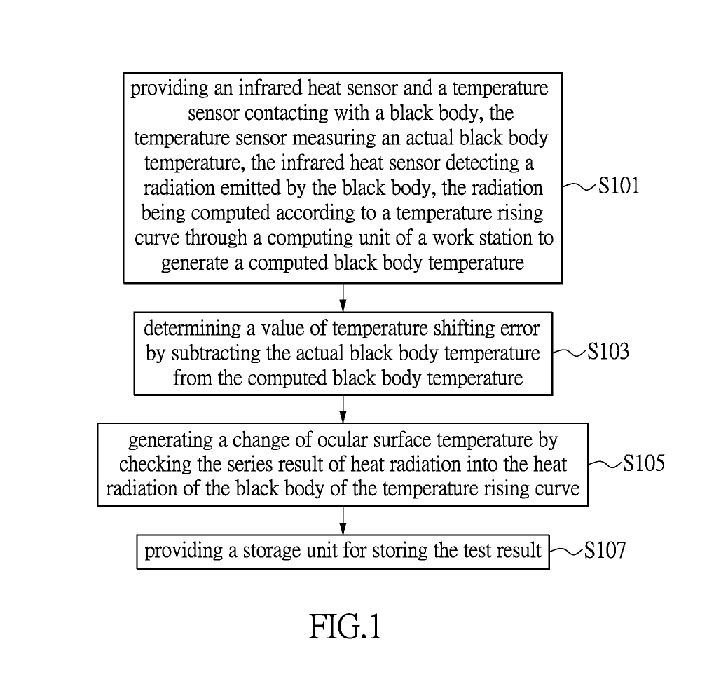

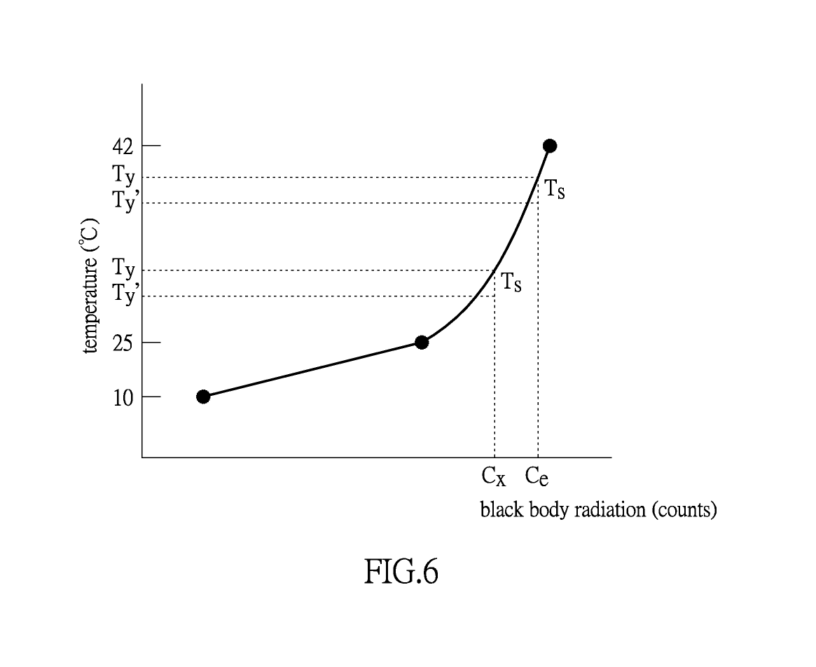

Please refer to FIGS. 1, 3, 5 and 6. The instant disclosure provides a method of determining temperature shifting error derived from radiation sensor and an apparatus thereof. The method includes the following step: Step S101: providing an infrared heat sensor 20 and a temperature sensor 31 buried in and contacting with a black body 30. The temperature sensor 31 measures an actual black body temperature Ty′. The infrared heat sensor 20 detects black body radiation Cx emitted by the black body 30 and the value of the black body radiation Cx is computed according to a provided temperature rising curve (FIG. 6) through a computing unit (not shown) of a work station 40 to generate a computed black body temperature Ty. Preferably, the computing unit computes according to the black body temperature rising curve in FIG. 6, mainly by the counts of the heat radiation (black body radiation Cx) emitted by the black body between 10 and 42 degree Celsius. The black body radiation emitted by the black body between 10 and 25 degree Celsius shows a linear trend, and the black body radiation emitted by the black body between 25 and 42 degree Celsius shows secondary curved correlation. In this way, when the infrared heat sensor 20 obtains heat radiation, e.g. the black body radiation Cx, between 10 and 42 degree Celsius, and the computed black body temperature Ty derived from calculation of black body radiation Cx into the temperature rising curve in FIG. 6, a value of temperature shifting error Ts can be determined by the following step: Step S103: subtracting the actual black body temperature Ty′ from the computed black body temperature Ty (Ty-Ty′). Calibration can be done according to the value of temperature shifting error Ts derived from (Ty-Ty′) in FIG. 6. Therefore, the infrared heat sensor 20 is free from ambient heat source that causes abnormal heat radiation.

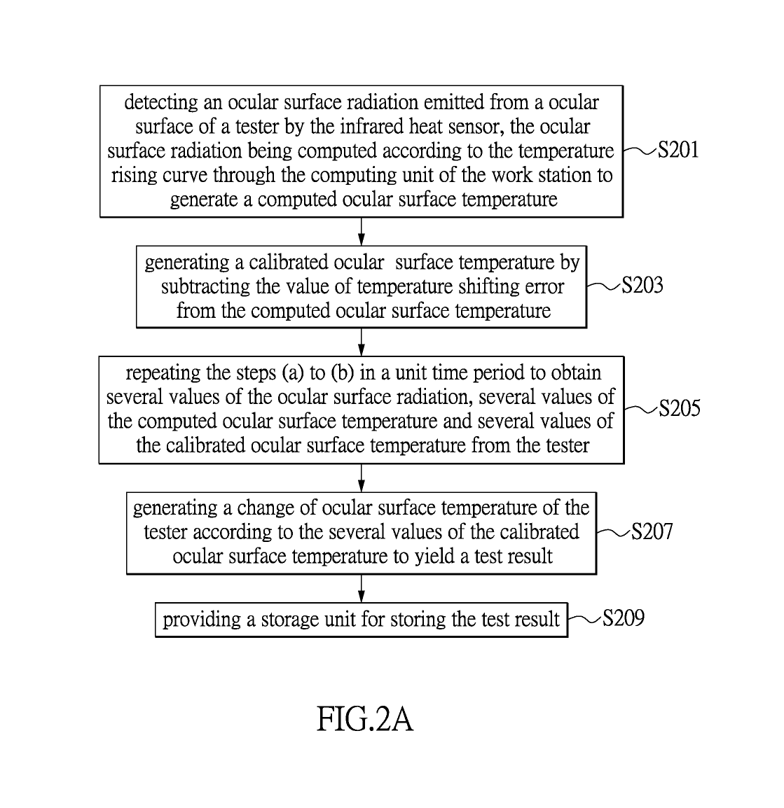

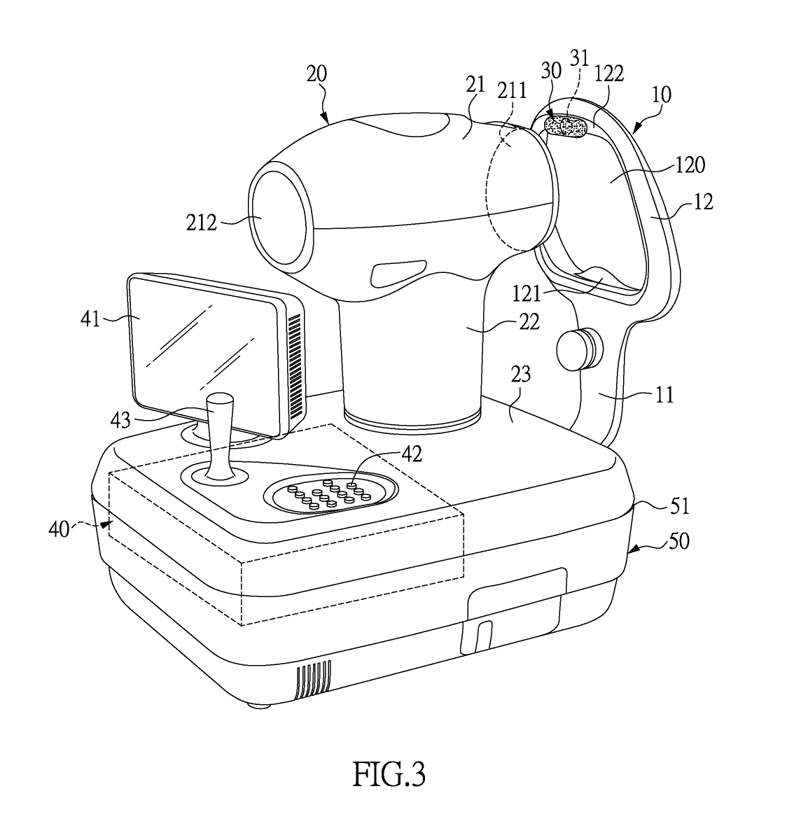

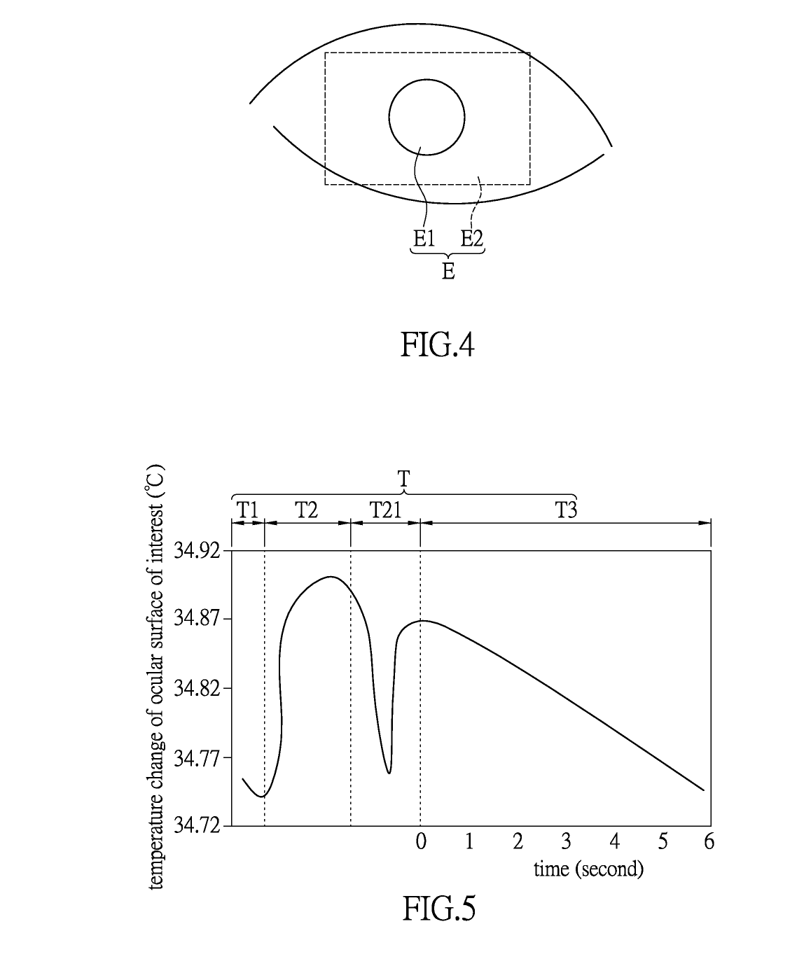

According to the above method of determining temperature shifting error derived from radiation sensor, please refer to FIGS. 2A, 3, 5 and 6, a method of measuring ocular surface temperature is also provided and comprising: Step S201: detecting an ocular surface radiation Ce emitted from an ocular surface E of a tester by the infrared heat sensor 20, the ocular surface radiation Ce being computed based on the temperature rising curve through the computing unit of the work station 40 to generate a computed ocular surface temperature Te. Step S203: generating a calibrated ocular surface temperature Te′ by subtracting the value of temperature shifting error Ts from the computed ocular surface temperature Te. Step S205: repeating the steps from S201 to S203 for several times in a unit time period to obtain several values of the ocular surface radiation, several values of the computed ocular surface temperature Te and several values of the calibrated ocular surface temperature Te′ from the tester. Step S207: generating a change of ocular surface temperature of the tester according to the several values of the calibrated ocular surface temperature Te′ to yield a test result. Step S209: a storage unit (not shown) is provided by the working station 40. The storage unit stores the test result. In order to prevent the infrared heat sensor 20 of the instant disclosure from ambient heat radiation interference, when using the instant disclosure, the infrared heat sensor 20 can be adjusted to a temperature range for detecting and displaying according to practical requirement which has an upper and lower limit. The upper and lower limit can be represented by different colors. For example, the upper limit may be red, the lower limit may be blue, and in between in upper and lower limit may be yellow or orange, such that the ocular surface F as shown in FIG. 4 can create a heat distribution graph (not shown) with different colors. The temperature of the test result should then fall into the temperature range. For example, as shown in FIG. 5, the temperature range is set between 34.92 and 34.72 degree Celsius, and the instant disclosure is not limited thereto. The temperature range is subject to change for practical requirement.

In addition, in the step S205, the method may further include providing a speech unit (not labeled). The speech unit is preferably disposed in the working station 40, and the instant disclosure is not limited thereto. As shown in FIG. 2B, the speech unit can guide the tester to do the next step. Step 301: instructing the tester to close the eyes, and the speech unit gives a subsequent instruction after the end of a first predetermined time period T1. The first predetermined time T1 may be 6 seconds. In other words, in the step S301, the tester is asked to close the eyes and rest for 6 seconds, but it is not limited thereto. After resting 6 seconds, step S303 carries on. Step S303: the speech unit instructs the tester to open the eyes, and the speech unit gives another subsequent instruction after the end of a second predetermined time period T2. The maximum value of the second predetermined time period T2 may be 1.5 second, but the instant disclosure is not limited thereto. After the tester opens the eyes for 1.5 second, step S305 is carried out. Step S305: the speech unit instructs the tester to blink once and then keep the eyes open. Next, the infrared heat sensor 20 measures the ocular surface temperature change of the tester within a third predetermined time period T3. After that, the step S307: the speech unit informs the testing the measurement is complete. Preferably, the third predetermined time is longer than 5 seconds. In the instant embodiment, the third predetermined time is 6 seconds. That is to say, the infrared heat sensor 20 can perform the measurement of ocular surface temperature of the tester in 6 seconds. The test result includes the test results since the first predetermined time period T1 to the second predetermined time period T2 and to the third predetermined time period T3.

The test results of values of the calibrated ocular surface temperature Te′ are stored in the storage unit and are computed by a computing unit (not shown) from the working station 40 to generate a graphical result. FIG. 5 shows the test result in graphical curve obtained by using the method of measuring ocular surface temperature of the instant disclosure and then computed by the computing unit. According to FIG. 5, the three longitudinal dotted lines divide the lateral axis into four blocks from the left to right. From the left to right are the first predetermined time period T1, the second predetermined time period T2, the blinking time period T21 and the third predetermined time period T3. At the first predetermined time period T1, the tester's eyes are closed, such that the result is relatively lower, approximately below 34.77 degree Celsius. At the second predetermined time T2, the temperature rises. In the blinking time period T21, the peak and trough are shown because of blinking. From the 0 second of the third predetermined time period T3, the ocular surface temperature gradually declines along the time (0 to 6 second). Meanwhile, the infrared heat sensor 20 can also film the eyes. The decline of the ocular surface temperature means the tear is evaporating. Because in the process of evaporation absorbs heat, the ocular surface temperature falls. Therefore, the declining trend in the third predetermined time period T3 can represent the trend of tear evaporation, and the condition of tear over the ocular surface can be understood. However, the instant disclosure truthfully displays and records the temperature without passing any judgment of diagnosis. What the medical implication associated with the declining temperature is left to be determined by relevant personnel.

Moreover, the first predetermined time period T1, the second predetermined time period T2 and the blinking time period T21 are the preparation time before actually measuring the ocular surface temperature. The sum of the preparation time and the third predetermined time period T3 is the unit time T. The eye closing, opening and blinking in the preparation time are all recorded and saved, such that the same basic state of the tester's eyes before each of the measurement can be ensured. The test result obtained in the third predetermined time period T3 is therefore more reliable.

Please refer to FIGS. 4 and 5. The ocular surface temperature change shown on the y axis of FIG. 5 may show the temperature change of cornea E1 as shown in FIG. 4. In other words, the method of measuring ocular surface temperature is not limited to the overall temperature of ocular surface E, it can also be used to measure a partial temperature such as the cornea E1 of the ocular surface E or the temperature of the conjunctiva E2 of the ocular surface E. Then, the temperature change of the cornea E1 or the conjunctiva E2 can be shown as the curve in FIG. 5 in a unit time T. Preferably, please refer to FIG. 4 showing the test result obtained from the method of the instant disclosure. The graphical result may be a plurality of heat distribution images (not shown) or the original ocular surface image (as shown in FIG. 4). The abovementioned filming can be displayed as heat distribution image or the original ocular surface (as shown in FIG. 4). The temperature change and distribution of all parts in the unit time T or the third predetermined time period T3 can be displayed. Therefore, within the third predetermined time period T3, it can be understood that which part of the ocular surface E reaches the lower temperature, and it can be told where the tear evaporates first on the ocular surface E.

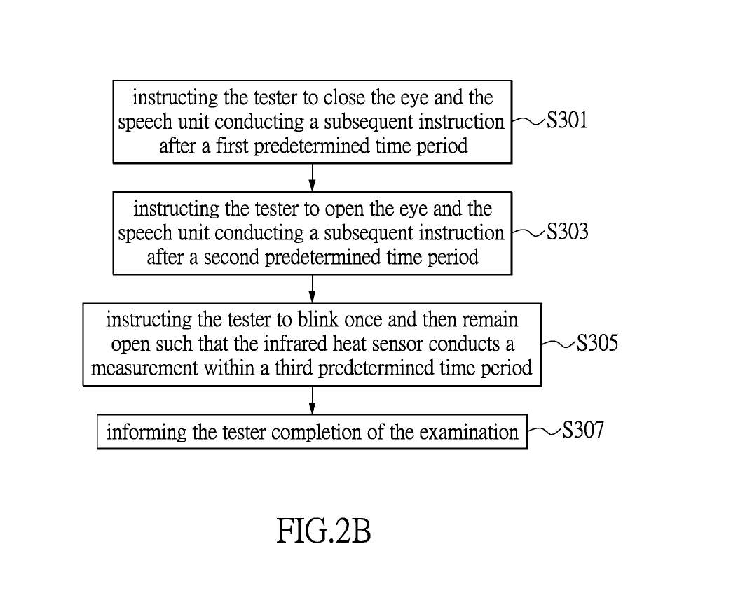

Please refer to FIG. 3. The instant disclosure further provides an apparatus to perform the above method of measuring ocular surface temperature. The apparatus to measure ocular surface temperature includes a supporting body 10, an infrared heat sensor 20, a black body 30, a temperature sensor 31 and a working station 40. The supporting body 10 can be used to support the head of the tester. More specifically, the support body 10 has an upwardly extending pole 11 stretching from a side portion (not shown) of a base 50. The pole 11 further extends to form a ring portion 12. A receiving space 120 defined by the ring portion 12 receives the head of the tester. The bottom and top portions of the inner circle of the ring portion 12 are a chin supporting portion 121 and a forehead abutting portion 122 respectively to accommodate the head of the tester in the ring portion 12.

The infrared heat sensor 20 and the supporting body 10 are arranged oppositely, such that the supporting body 10 is positioned in a predetermined view field of the infrared heat sensor 20. Preferably, the infrared heat sensor 20 further includes a sensor main body 21. Inside the sensor main body 21 is an infrared sensor component (not labeled). The infrared heat sensor may be a heat sensitive sensor, for example, thermopile, pyroelectric component or bolometer. In the instant embodiment, microbolometer is used as the heat sensitive component. One end of the sensor main body 21 is an image capture end 211, and the other end of the sensor main body 21 is a view window 212. The image capture end 211 and the supporting body 10 are arranged oppositely, while the view window 212 is positioned toward an operator (not shown), such that the operator can see the image captured by the infrared heat sensor 20 from the image capture end 211 via the view window 212. The sensor main body 21 extends downwardly to form a neck portion 22 and a secondary base 23. The secondary base 23 resembles a platform and is disposed on a top platform 51 of the base 20. Preferably, the infrared heat sensor 20 can be slidably disposed on the top platform 51.

The black body 30 is a black plate-like object. In FIG. 3, it is schematically shown as a plate with black dots. The black body 30 is disposed on the forehead abutting portion 122 of the supporting body 10, and it is located within the view field of the infrared heat sensor 20. Therefore, when the infrared heat sensor 20 measures the ocular surface temperature of a tester, the black body 30 acts as a reference for calibration. In FIGS. 3 and 6, the temperature sensor 31 is buried in the black body 30 and contacted with the black body 30 so that the temperature sensor 31 is able to measure an actual black body temperature Ty′ from the black body 30.

Please refer to FIGS. 1, 2A, 2B, 3 and 6. The working station 40 is preferably disposed on the secondary base 23 (but not limited thereto) and upwardly electrically connected to the infrared heat sensor 20 by the secondary base 23. The working station 40 also includes a computing unit (not shown) and a storage unit (not shown). The infrared heat sensor 20 is used to detect a value of radiation emitted from the black body 30 and the computing unit turns the value of radiation into a computed black body temperature Ty according to a temperature rising curve. Consequently, a value of temperature shifting error Ts is determined by subtracting the actual black body temperature Ty′ from the computed black body temperature Ty. Furthermore, the storage unit of the working station 40 stores the result of temperature shifting error Ts. The temperature shifting error Ts can be used for correctly measuring the ocular surface temperature and other applications as the two aforementioned method introduced. The working station 40 further includes a display 41, a function key module 42 and a motion control unit 43. The user can use the motion control unit 43 to adjust the position of the infrared heat sensor 20, such that the infrared heat sensor 20 can slide to appropriate position on the top platform 51 of the base 50. Then the infrared heat sensor 20 can aim at one eye of the tester's and focus on the ocular surface. After that, the operator can use the function key module 42 to execute “image property switch”. The image property may be divided into three image property: color palette, temperature range and temperature range center. The color palette is responsible for the tone modification, the temperature range center can define the central point of the ocular surface temperature range, and the temperature range defines the temperature range shown in the image. Take FIG. 5 for example. The highest temperature of y axis in FIG. 5 is 34.92 degree Celsius, and the lowest temperature is 34.72 degree Celsius. When showing the heat distribution image (not shown), the region of the highest temperature is shown in red and the region of the lowest temperature is shown in blue. The display 41 shows the graphical result after measurement and the adjusting status done through the function key module 42. In the process of examination, the change of ocular surface tear in the time of examination can be recorded by function key module 42 giving a filming order.

The infrared heat sensor 20 of the instant disclosure has the black body 30 in its predetermined view field. Overall, the black body 30 is viewed as a calibration reference when the infrared heat sensor 20 measures the ocular surface temperature so as to help obtain an accurate ocular surface temperature test result. Furthermore, the test result is converted and computed by the computing unit of the working station 40 to obtain a graphical result, and the in the process of measuring, it is also recorded by filming. The graphical result includes heat distribution image, conventional ocular surface temperature and ocular surface temperature change curve of temperature against time. The apparatus to measure ocular surface temperature of the instant disclosure can fully support the abovementioned method of measuring ocular surface temperature. The instant disclosure is not intrusive and the error rate is relatively low compared to conventional infrared detector.

The descriptions illustrated supra set forth simply the preferred embodiments of the instant disclosure; however, the characteristics of the instant disclosure are by no means restricted thereto. All changes, alternations, or modifications conveniently considered by those skilled in the art are deemed to be encompassed within the scope of the instant disclosure delineated by the following claims.

What is claimed is:

1. A method of determining temperature shifting error derived from radiation sensor comprising:
(A) providing an infrared heat sensor and a temperature sensor contacting with a black body, the temperature sensor measuring an actual black body temperature, the infrared heat sensor detecting a radiation emitted by the black body, the radiation being computed according to a temperature rising curve through a computing unit of a work station to generate a computed black body temperature; and
(B) determining a value of temperature shifting error by subtracting the actual black body temperature from the computed black body temperature.

2. A method of measuring ocular surface temperature according to claim 1, comprising:
(a) detecting an ocular surface radiation emitted from a ocular surface of a tester by the infrared heat sensor, the ocular surface radiation being computed according to the temperature rising curve through the computing unit of the work station to generate a computed ocular surface temperature;
(b) generating a calibrated ocular surface temperature by subtracting the value of temperature shifting error from the computed ocular surface temperature.

3. The method of measuring ocular surface temperature according to claim 2, further comprising:
repeating the steps (a) to (b) in a unit time period to obtain several values of the ocular surface radiation, several values of the computed ocular surface temperature and several values of the calibrated ocular surface temperature from the tester; and
generating a change of ocular surface temperature of the tester according to the several values of the calibrated ocular surface temperature to yield a test result.

4. The method of measuring ocular surface temperature according to claim 2, wherein the step (A) further comprising:
adjusting a temperature range that the infrared heat sensor is effective such that the test result falls in the temperature range.

5. The method of measuring ocular surface temperature according to claim 2, wherein further comprising:
providing a speech unit, the speech unit guiding the tester by:
instructing the tester to close the eye and the speech unit conducting a subsequent instruction after a first predetermined time period;
instructing the tester to open the eye and the speech unit conducting a subsequent instruction after a second predetermined time period;
instructing the tester to blink once and then remain open such that the infrared heat sensor conducts a measurement within a third predetermined time period; and
informing the tester completion of the examination.

6. The method of measuring ocular surface temperature according to claim 5, wherein the first predetermined time period is 6 second.

7. The method of measuring ocular surface temperature according to claim 5, wherein the second predetermined time period is 1.5 second.

8. The method of measuring ocular surface temperature according to claim 5, wherein the predetermined time period is 6 second.

9. The method of measuring ocular surface temperature according to claim 3, wherein computing unit continues to process the test result to generate a graphical result.

10. The method of measuring ocular surface temperature according to claim 9, wherein the graphical result includes a plurality of heat distribution images to show the temperature change and temperature distribution of each portion of the ocular surface in the unit time.

11. The method of measuring ocular surface temperature according to claim 9, wherein the graphical result is a curve graph showing the temperature change of each portion of the ocular surface in the unit time.

12. An ocular surface temperature measurement apparatus comprising at least:
a supporting body for supporting a head;
an infrared heat sensor disposed opposite to the supporting body such that the supporting body is positioned in a predetermined view field of the infrared heat sensor;
a black body disposed on the supporting body and in the predetermined view field;
a temperature sensor buried in and contacted with the black body, wherein the temperature sensor measures an actual black body temperature from the black body; and
a working station electrically connected to the infrared heat sensor, the working station having at least a computing unit and a storage unit,
wherein the infrared heat sensor detects a value of radiation emitted from the black body and the computing unit turn the value of radiation into a computed black body temperature according to a temperature rising curve;
wherein a value of temperature shifting error is determined by subtracting the actual black body temperature from the computed black body temperature.

13. The ocular surface temperature measurement apparatus according to claim 12, wherein the infrared heat sensor is slidably mounted on a top platform of a base.

14. The ocular surface temperature measurement apparatus according to claim 12, wherein the working station further includes a display, a function key module and a motion control unit.
